(19) United States
(12) Patent Application Publication
(10) Pub. No.: US 2026/0216953 A1
(43) Pub. Date: Jul. 30, 2026

(54) PRINTERS FOR MANUFACTURING CONSTRUCTS FOR REGENERATIVE MEDICINE

(71) Applicant: UNIVERSITY OF CONNECTICUT, Farmington, CT (US)

(72) Inventors: Ali TAMAYOL, Providence, RI (US); Mohamadmahdi SAMANDARI, Newington, CT (US); Farnoosh SAEEDINEJAD, Hartford, CT (US); Jacob P. QUINT, New Britain, CT (US); Amir SEYEDSALEHI, Farmington, CT (US)

(21) Appl. No.: 19/145,696

(22) PCT Filed: Jan. 5, 2024
(86) PCT No.: PCT/US2024/010556

§ 371 Date: Jul. 3, 2025

Related U.S. Application Data
(60) Provisional application No. 63/437,351, filed on Jan. 5, 2023.

(51) Int. Cl. B29C 64/124; A61L 27/52; A61L 27/56; B29C 64/314; B29K 105/00; B29K 105/04; B29L 31/00; B33Y 10/00; B33Y 30/00

(52) U.S. Cl. CPC B29C 64/124; A61L 27/52; A61L 27/56; B29C 64/314; B33Y 10/00; B33Y 30/00; B29K 2105/0061; B29K 2105/045; B29L 2031/7532

(57) ABSTRACT

Disclosed herein is a printer for manufacturing a construct for use as a prosthetic, the printer comprising a housing; a piston that is operative to reciprocate in the housing to extrude a volume of a hydrogel; and a stage comprising a porous screen and an inlet port disposed downstream of the housing; where the inlet port is operative to introduce air into the hydrogel; and where the porous screen is operative to reduce air bubble sizes in the hydrogel.

### CROSS-REFERENCE TO RELATED APPLICATIONS

This disclosure claims priority to U.S. Provisional Application No. 63/437,351 filed on Jan. 5, 2023, the entire contents of which are hereby incorporated by reference.

### BACKGROUND

This disclosure relates to a printer for manufacturing a construct for regenerative medicine. In particular, this disclosure relates to a hand-held printer for manufacturing the construct. This disclosure also relates to a method for manufacturing a construct from a hydrogel for purposes of regenerative medicine.

Regenerative medicine is a broad field that includes tissue engineering but also incorporates research on self-healing—where the body uses its own systems, sometimes with the assistance of a foreign biological material to recreate cells and rebuild tissues and organs. Foreign biological materials (hereinafter “biological substitutes”) in regenerative medicine refer to materials or constructs that are used to replace or repair damaged or lost tissues and organs within the body. These biological substitutes aim to harness the body's natural regenerative capacities or provide support for regenerative processes.

As scientists develop biological substitutes for regenerative medicine, tissue engineering has continued to evolve. Most human and animal tissues are anchorage-dependent, residing in a solid matrix referred to as an “extracellular matrix (ECM)”. The ECM is a complex three-dimensional network of proteins, carbohydrates, and other molecules that provide structural and biochemical support to the cells within tissues and organs. The ECM is also referred to as a “construct”.

Hydrogels are often used to generate such constructs because of their tunable properties. Hydrogels are networks of hydrophilic polymer chains with nano- to micro-scale porous structures. Their porous structures allow gas and small molecules to diffuse throughout the network, providing a nurturing environment for the encapsulated cells.

However, large sized hydrogels, which are often manufactured by methods including molding, demonstrate mass and transport limitations which inhibit cell infiltration. This impairs the viability and functionality of encapsulated cells. It is therefore desirable to develop methods and hand-held printers for manufacturing such constructs, which do not inhibit cell infiltration.

### SUMMARY

Disclosed herein is a printer for manufacturing a construct for use as a prosthetic, the printer comprising a housing; a piston that is operative to reciprocate in the housing to extrude a volume of a hydrogel; and a stage comprising a porous screen and an inlet port disposed downstream of the housing; where the inlet port is operative to introduce air into the hydrogel; and where the porous screen is operative to reduce air bubble sizes in the hydrogel.

Disclosed herein too is a printer for manufacturing a construct for use as a prosthetic, the printer comprising a first syringe; a second syringe; a three-way valve in fluid communication with and located downstream of the first syringe and the second syringe; and a porous screen located in the three-way valve in a fluid pathway between the first syringe and the second syringe; where the mesh is operative to reduce a size of air bubbles contained in a hydrogel or a bioink that is ejected from the first syringe into the second syringe.

Disclosed herein too is a method of manufacturing a construct, the method comprising blending a hydrogel with air to form an aerated hydrogel; transmitting the aerated hydrogel through a plurality of porous screens to form a foam; disposing the foam on a substrate; and curing the foam with electromagnetic radiation to form the construct.

### BRIEF DESCRIPTION OF THE FIGURES

FIG. 1A depicts a hand-held printer that may be used in the printing of constructs for tissue regeneration;

FIG. 1B depicts the size reduction in air bubbles as they are transported through several stages that lie downstream of the nozzle; where at least some of the stages may contain a porous screen;

FIG. 2 depicts one embodiment for producing a foam by a process of repetitive bubble splitting that may be performed through back-and-forth flow of air and hydrogel through a micromesh installed between two syringes;

FIGS. 3A-3D depicts the different modules that can be combined with the hand-held printer of the FIGS. 1A and 2;

FIGS. 4A-4D are depictions of exemplary microfluidic nozzles;

FIG. 5 is a depiction of an exemplary automated printer, where the hand-held printer of FIG. 1A or 3A-3D is reversibly attached to a robot arm that is controlled via a microprocessor;

FIG. 6 is a bar chart that depicts the fluorescence intensity measurements versus time for the different foams;

FIG. 7A is a graph that depicts wound closure rate in mice for the different foams as a function of time; and

FIG. 7B is a bar graph that shows that the wound closure data reveals that at all time points after day 5, the control and one-step printed foam groups shows significantly better wound closure compared with the bulk hydrogel group.

### DETAILED DESCRIPTION

Disclosed herein is a hand-held printer along with a method of using the hand-held printer to manufacture a construct that can be used as a prosthetic on and/or in the body of a living being to facilitate tissue regeneration. In one embodiment, the hand-held printer comprises a piston that is in reciprocating contact with a cylindrical chamber that comprises a number of reversibly attachable sections (hereinafter stages). Each stage comprises a porous screen that facilitates a reduction in the size of voids present in a material (such as for example, a hydrogel) as it travels through the hand-held printer. This results in a highly porous liquid foam that can be extruded from the hand-held printer and subjected to curing to produce a solid foam (hereinafter construct or scaffold). In an embodiment, the highly porous liquid foam has a consistency similar to that of whipping cream or shaving cream.

Bioprinting technologies have received significant attention in tissue engineering due to their ability to fabricate complex constructs by precise deposition of bioinks to form 3D constructs. Among various bioprinting approaches, extrusion-based bioprinting has attracted more attention due to its compatibility with a wide range of bioink viscosities, decent reliability, and capability of large-scale construct fabrication with clinically relevant dimensions. Extrusion-based bioprinters also allow the fabrication of constructs that contain multiple materials. However, the quality of printing and the system reproducibility heavily depends on the characteristics of the bioink.

A bioink refers to a specialized type of ink used in 3D bioprinting; a cutting-edge technology that enables the fabrication of three-dimensional structures composed of living cells. A bioink may comprise living cells, biomaterials, other components that provide structural support, nutrients, a conducive environment for cell growth, or a combination thereof. The bioink may be used to create a printable material that mimics the natural extracellular matrix (ECM) found in tissues.

Various biomaterials have been implemented for the 3D bioprinting of tissue engineering scaffolds. Among them, hydrogels have been broadly applied as bioinks due to their similarity to the native extracellular matrix (ECM) and tunability of their properties. Hydrogels are networks of hydrophilic polymer chains with nano- to micro-scale porous structures. Their porous structures allow gas and small molecules to diffuse throughout the network, providing a nurturing environment for the encapsulated cells. However, by increasing the bulk size of the hydrogel construct, the diffusion capability is severely diminished, resulting in cell-death within the depth of the construct. In addition, poor cell infiltration and spreading within these hydrogels further limits early vascularization and innervation, thus impairing the viability and functionality of encapsulated cells. Incorporating micro to millimeter-scale channels within the hydrogel, through multi-material extrusion 3D bioprinting, is a possible resolution. However, the engineered porosity using these techniques can negatively influence the mechanical properties, fidelity, and structural stability of the final scaffold. Therefore, a high concentration of the hydrogel bioink may have to be utilized to stabilize the printed constructs, which in turn limits cell spreading, migration, and tissue integration. While pre-vascularization and pre-innervation strategies can also be implemented (to facilitate the viability and functionality of the encapsulated cells), these strategies make the 3D bioprinting and subsequent tissue implantation highly complex.

3D bioprinting technologies have experienced rapid growth over the past decade. Extrusion-based bioprinting has emerged as the preferred technique among various methods employed in bioprinting. Hydrogels are frequently chosen as the biomaterial foundations of bioinks due to their close resemblance to the ECM and their ability to provide customizable properties. Gelatin methacrylol (GelMA), a crosslinkable biomaterial, may be used for tissue engineering applications owing to its biocompatibility, bioadhesivity, and the ease with which it can undergo photocross-linking. Its base protein, gelatin, is obtained from hydrolyzed collagen, a major component of the extracellular matrix in various tissues. The modification of gelatin to create GelMA involves the addition of methacryloyl groups. This modification allows the gelatin to cross-link through free radical polymerization when in the presence of photoinitiators that are exposed to ultraviolet (UV) or blue light, forming a stable and mechanically robust hydrogel. The cross-linking process is essential in bioprinting applications as it helps to maintain the structural integrity of the printed construct.

The modification of gelatin to create GelMA involves the addition of methacryloyl groups. This modification allows the gelatin to cross-link when exposed to ultraviolet (UV) light, forming a stable and mechanically robust hydrogel. The cross-linking process is essential in bioprinting applications as it helps to maintain the structural integrity of the printed construct.

Nevertheless, the restricted diffusion of nutrients, oxygen, and waste within the small microporous hydrogel network diminishes cellular activity within conventional GelMA-based constructs, particularly when constructing scaffolds with clinically relevant dimensions. The small pore size in the GelMA structure further hampers cell infiltration, expansion, and consequently, the ultimate integration of the tissue. To address the challenges associated with GelMA bioinks, multiscale porosity has been introduced into the hydrogel precursor through a foaming process involving high-speed stirring with a homogenizer.

A substantial enhancement in the cell permissibility and regenerative capacity has been achieved using this approach with bioprinted GelMA constructs. However, the applicability of this method for in-situ bioprinting is constrained due to its multi-step nature, lacking the capability for simultaneous foam generation and printing. Additionally, the mechanical agitation process involves numerous manual steps, compromising its repeatability.

To resolve these challenges, microfluidic nozzles have been developed and integrated into an in-situ printer to simultaneously generate highly controllable foams that can be printed instantaneously. This process is highly repeatable and provides good control over the porosity and pore size distribution of the printed foam by modifying the configuration of the nozzles or parameters of the in-situ printer. Details of the hand-held printer as well as those pertaining to the nozzles are provided later.

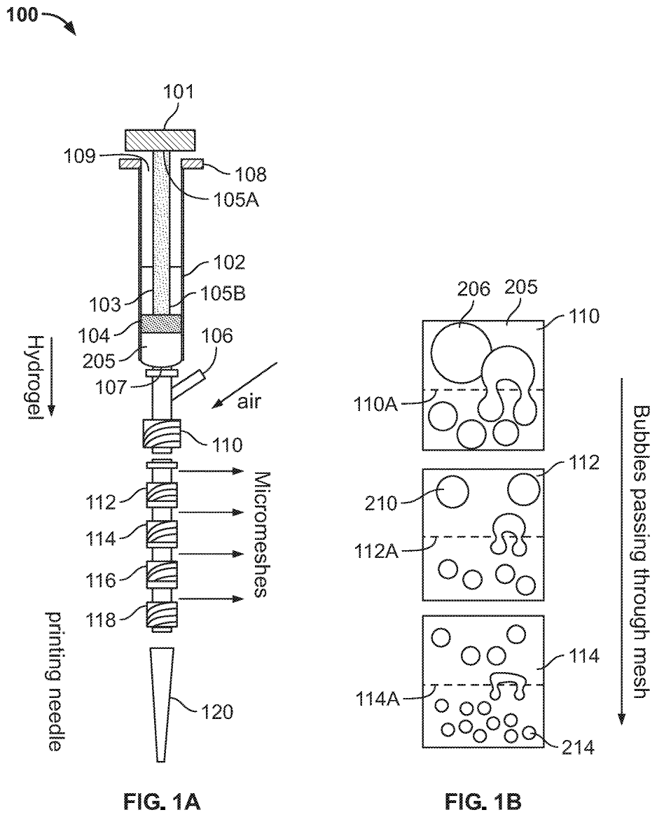

FIG. 1A depicts a hand-held printer 100 that may be used in the printing of constructs for tissue regeneration. In an embodiment, the hand-held printer 100 comprises a syringe that is located upstream of a plurality of stages, each stage of which comprises a porous screen that facilitates a size reduction in air bubbles contained in an injected hydrogel.

The hand-held printer 100 comprises a chamber 102 (also referred to herein as a “housing”) in which a reciprocating shaft 103 is disposed. The reciprocating shaft 103 has a distal end 105B and a proximal end 105A. The reciprocating shaft 103 is fixedly attached to a piston 104 at its distal end 105B. The proximal end 105A of the reciprocating shaft 103 may be in contact with a motor (not shown) or may be in contact with a plunger 101. The hand-held printer 100 is operable by a human being in a manner similar to the operation of an injection syringe.

The piston 104 contacts an inner surface of the chamber 102 forming a seal with the inner surface. The chamber 102 is in contact with a nozzle 107 at its distal end. The portion of the chamber 102 between the piston 104 and the nozzle contains a hydrogel 205 that may be injected through the nozzle 107. The hydrogel 205 may be introduced into the chamber from an opening 109 located close to the proximal end 105A of the shaft 103. The piston 104 along with the shaft 103 is also introduced into the chamber 102 through the opening 109. The hydrogel 205 (either in pure form or in a mixture with a solvent) is introduced into the chamber 102 prior to the introduction of the piston 104 and shaft 103. The chamber 102 is preferably cylindrical and defines a housing for accommodating the piston, wherein the inner surface of the cylinder provides a sliding interface for the reciprocal movement of the piston 104. The piston head includes sealing means to create a substantially fluid-tight seal between the piston and the inner surface of the cylinder, thereby facilitating the compression and expansion of fluid within the chamber during the reciprocating motion. A driving mechanism (e.g., such as a stepper motor) (not shown) is in operative communication with the piston rod and is adapted to impart reciprocating motion to the piston within the cylinder.

The distal end of the syringe is located at the farthest end of the syringe from the opening 109, while the proximal end of the syringe is located at the opening 109 of the syringe. The distal end of the syringe is located opposite to the proximal end.

Downstream of the nozzle 107 are a plurality of stages arranged in series—a first stage 110, a second stage 112, a third stage 114, a fourth stage 116, and so on. A printing needle 120 is located downstream of the last stage and in contact with it. The hydrogel 205 from the chamber 102 travels through the nozzle 107 through each successive stage 110, 112, 114, and 116 before being ejected from the printing needle 118 on a substrate (not shown). The hydrogel 205 on the substrate may be cured using thermal energy or electromagnetic radiation. The electromagnetic radiation may include visible light, ultraviolet light, infrared radiation, radio frequency radiation, microwave radiation, xray radiation and/or electron beam radiation. Other forms of crosslinking due to enzymatic or free-radical crosslinking are also contemplated herein.

One or more stages 110, 112, 114 and 116 are provided with a porous screen to facilitate the division of air bubbles that are entrapped in the hydrogel 205 as seen in the FIG. 1B. Each stage may also be provided with a side inlet port 106, through which air may be injected into the extrudate emanating from the nozzle 107. The air mixes with the hydrogel to form an aerated hydrogel. The air exists in the form or air bubbles dispersed in the mass of the hydrogel. In an embodiment, the air is replaced with an inert gas.

In the FIG. 1A, only the first stage 110 is provided with the side inlet port 106. When each stage is provided with an inlet port, other components, and ingredients such as initiators, photoinitiators, crosslinking agents, fillers, or other additives may be added to the hydrogel 205.

FIG. 1B depicts how the air bubbles 206 introduced as pressurized air via side inlet port 106 are reduced in size during each contact with successive screens 110A, 112A and 114A. The air may be introduced into the port 106 via a motor (not shown). The air flow to hydrogel extrusion rate ratio is 300 to 2000, preferably 500 to 1600 and more preferably 700 to 1500.

The hydrogel 205 containing large air bubbles 206 in stage 110 passes through screens 110A, 112A (in stage 112) and 114A (in stage 114). The air bubbles 206 are reduced in size with each contact with a screen becoming smaller (see air bubbles 210 after contact with the second screen 112A) and smaller (see air bubbles 214 after contact with the third screen 114A).

The introduction of air bubbles and their reduction in size (by transporting the hydrogel through several stages with porous screens) results in the formation of a foam. The foam is called a bioink when it contains therapeutic cells. The bioink may be used for printing the construct. The foam becomes more porous with passage through each porous screen until it is ejected from the printing needle 118. The foam upon ejection from the printing needle is crosslinked using thermal energy or radiation.

In an embodiment, the successive porous screens 110A, 112A, 114A, and so on, can each have the pores of the same sizes and the same amount of porosity, or alternatively, each successive porous screen can have pores of a different average size when compared with a preceding screen. In an embodiment, each successive screen located downstream of the nozzle may have smaller pores than those of a preceding screen.

When the pores of each successive screen are smaller than those of the preceding screen, each successive screen may have a different porosity from those of the preceding screen. In an embodiment, as the average pore size in successive screens (in the downstream direction) become smaller and smaller, the amount of porosity in each screen may get larger and larger. The pores in the screens may be periodically spaced or aperiodically spaced.

In an embodiment, therapeutic cells may be incorporated into the hydrogel contained in the chamber 102. By encapsulating therapeutic cells in the hydrogel before the incorporation of air, a homogeneous foam bioink with cells present therein maybe produced. The flow of foam can be used for printing of the macroporous construct. This one step method of foam printing can be used on any bioprinter, just by integration of the microfluidic two-way inlet (for hydrogel and air), the micromesh-incorporated connectors, and a printing needle (See FIG. 1). The number of micromesh-incorporated connectors can be adjusted for optimized foam structure and cellular viability (when using cellular bioinks). This hand-held printer can be operated in an automated fashion and can be controlled by one or two servo motors (not shown) to automatically move the pistons before starting the printing process. For example, one servo motor can control the movement of the piston to transport the hydrogel through the different stages, while another piston can control the amount of air charged through the inlet port 106.

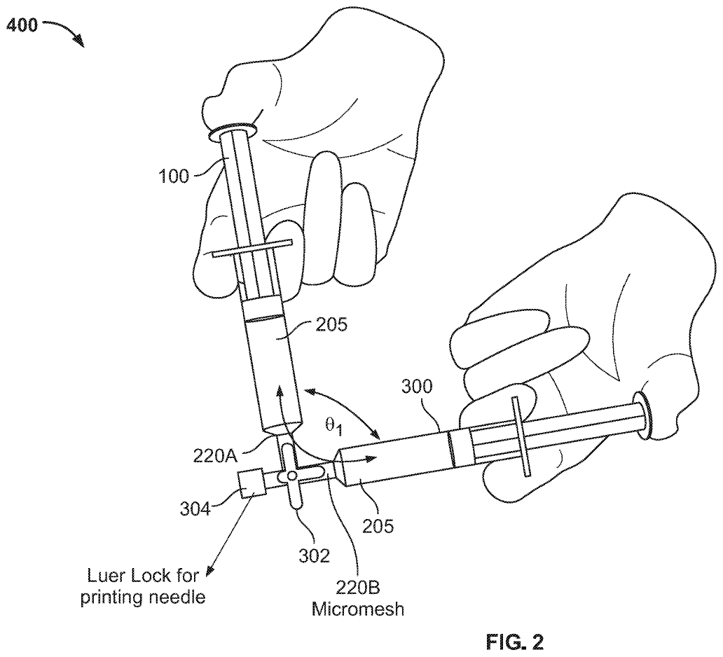

FIG. 2 depicts one embodiment for producing a foam by a process of repetitive bubble splitting that may be performed through back-and-forth flow of air and hydrogel through a micromesh installed between two syringes. The hand-held printer 400 comprises two syringes 100 and 300, each containing a controlled amounts of hydrogel and air and are connected using a micromesh-incorporated three-way valve.

In an embodiment, the printer 400 comprises a first syringe 100 and a second syringe 300. A three-way valve 302 is in fluid communication with and located downstream of the first syringe and the second syringe. One or more meshes (not shown) are located in the three-way valve in a fluid pathway between the first syringe 100 and the second syringe 300. The mesh is operative to reduce a size of air bubbles contained in a hydrogel that is ejected from the first syringe into the second syringe. The three-way valve is used to permit the air and hydrogel contained in the respective chambers of the syringe to be injected back-and-forth between the two syringes. When the hydrogel-air mixture has reached the right consistency, the valve 302 is rotated so that the foam is discharged from the hand-held printer 400 onto a substrate to undergo further processing.

In another embodiment, each syringe 100 and 300 contains a micromesh 220A and 220B respectively incorporated into the nozzle at the distal end. In a preferred embodiment, only one of the syringes 100 and 300 contains a micromesh located at the distal end of the syringe. A three-way valve 302 (with an outlet 304) is used to permit the air and hydrogel contained in the respective chambers of the syringe to be injected back-and-forth between the two syringes. When the hydrogel-air mixture has reached the right consistency, the valve 302 is rotated so that the foam is discharged from the hand-held printer 400 onto a substrate to undergo further processing (e.g., crosslinking).

In using the hand-held printer 400 of the FIG. 2, the air and hydrogel contained in each syringe are first mixed to form a homogeneous uniform foam by repetitive splitting of air bubbles in the hydrogel. Each time the hydrogel (with the air entrapped therein) is transported across one of the micromeshes, the air bubbles are reduced in size. When the foam has achieved the desired consistency, one of the first syringe or the second syringe is replaced with a third syringe containing a high concentration of a therapeutic cell solution. The micromesh is also removed with the first or second syringe. The therapeutic cells are mixed easily with the foam through back-and-forth motion of the pistons (See FIG. 1A). Removing the mesh from the pathway is desirable to reduce the shear stress applied to the cells and therefore to increase the biocompatibility of the process. A printing needle may be connected to the outlet 304 of the three-way valve and the syringe containing cell-laden foam bioink may be used for bioprinting.

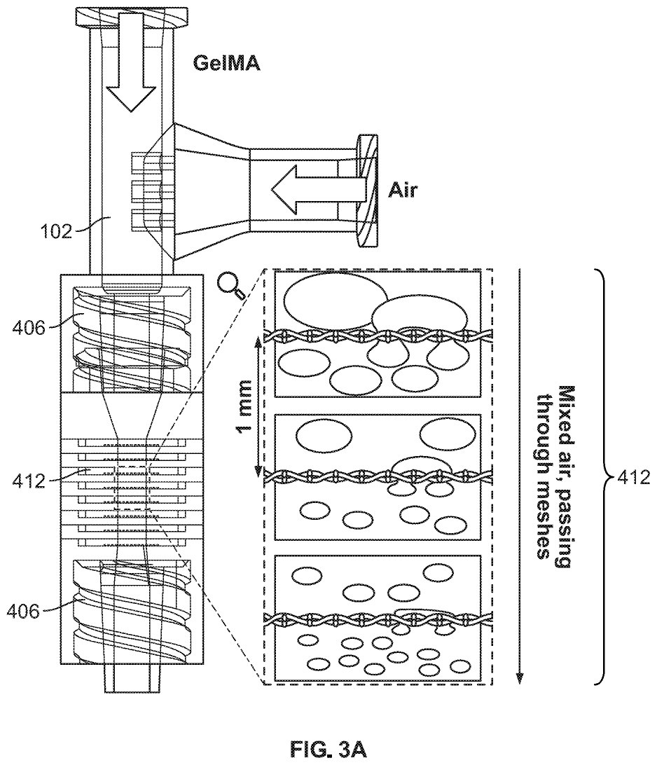

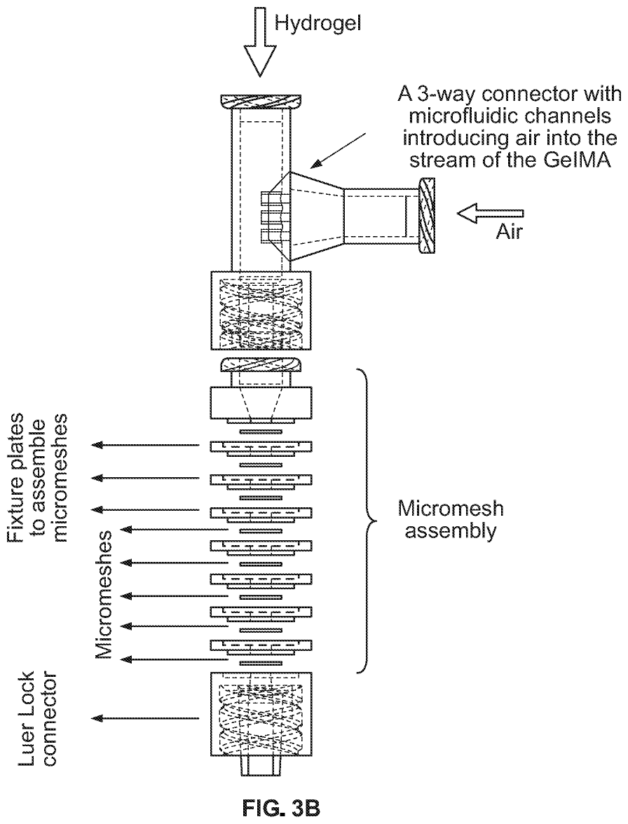

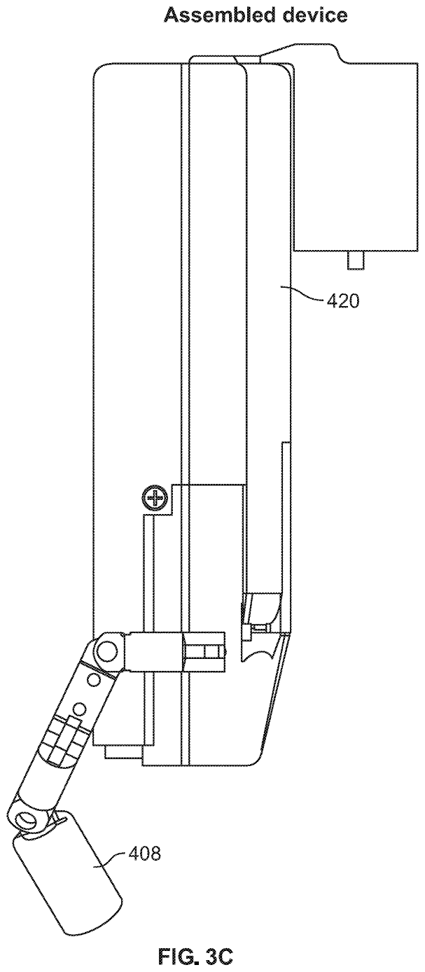

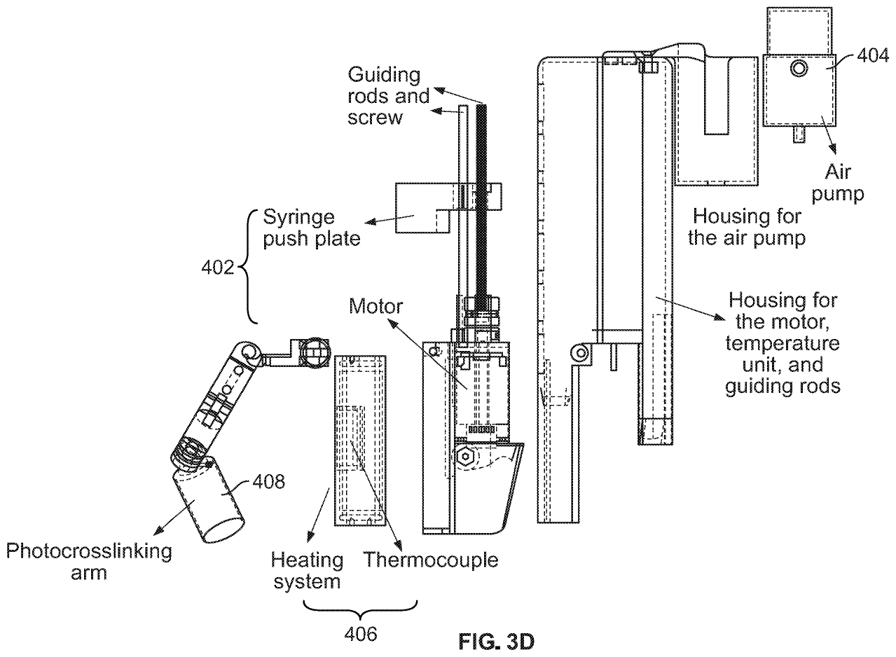

In yet another embodiment, the hand-held in-situ printer 100 of the FIGS. 1A and 2 may be combined with a plurality of additional integral modules. This embodiment will be discussed primarily with reference to the FIG. 1A. These additional integral modules include a motorized extrusion system 402, an air pump 404, a temperature control system 406 encompassing the chamber (that contains the hydrogel or the bioink), and a light photocuring system 408. These modules can be contained in a housing 420 (See FIGS. 3A, 3B, 3C and 3D).

FIGS. 3A, 3B, 3C and 3D depict the different modules that can be combined with the hand-held printer 100 or 400 of the FIGS. 1A and 2 respectively. FIG. 3B is a detailed version of the printer of the FIGS. 1A and 3A with the components spaced apart for easier viewing. FIGS. 3C and 3D show side views of the modular assembly and the components of the modular assembly respectively.

The modular design facilitates the fast and easy assembly and disassembly of the printer 100 and also facilitates an expeditious change of hydrogel chamber if such a change is needed during a printing process. Additionally, it can be useful in the case of any modification to the system for using other sizes of chambers, different motor types or any future upgrades, and bypasses the need for redesigning the whole system. The assembled in-situ printer may be very compact such that it is usable with only one hand, while the other hand can remain at ease.

The motorized extrusion system 402 comprises a motor that facilitates extrusion of the hydrogel from the chamber 102 (See FIGS. 1 and 3A-3D). In an embodiment, the motorized extrusion system 402 comprises a stepper motor that facilitates controlled precise extrusion of the hydrogel or hydrogel bioink, at a consistent speed, from the chamber 102. The rotation from the stepper motor is transmitted to the chamber through the linear guide rail system (see FIG. 3D) utilizing rolling bearings (not shown) and precision shafts (not shown).

The air pump 404 injects air into the hydrogel via an air pump that may be controlled by a DC motor. The air pump injects air into the hydrogel to facilitate the formation of a foam or a bioink. In an embodiment, a rotational speed of the stepper motor and the air pump's DC motor may be controlled through Pulse Width Modulation (PWM). Their speed and the amounts (of hydrogel and air) charged to the printer may therefore be controlled by modifying the values of the corresponding PWM functions via a microprocessor (not shown), as needed. The hydrogel extrusion rate and the generated air flow rate for any given PWM values in the programming code is measured and calibrated.

These measurements are used to calibrate the motorized extrusion system 402 and the air pump 404. Calibration curves may be generated and used to precisely control the rates of hydrogel extrusion and air flow. For in-vitro or in-vivo experiments, an air filter (not shown) may be incorporated between the mini air pump and the air inlet in the microfluidic nozzles, to make sure the introduced air is sterile. The air pump system in the in-situ printer is used only for printing foams, using the proposed microfluidic nozzles, and can be removed/disabled in case of using the in-situ printer for regular hydrogel/bioink printing with one inlet nozzles.

The temperature control system 406 ensures that the hydrogel in the chamber 102 will be kept at an optimum temperature for the whole duration of printing. The temperature control system 406 comprises a thermocouple (not shown) and a digital controller (not shown). The thermocouple measures temperature of the hydrogel in the chamber 102. The temperature control system comprises a heater (not shown) that is in electrical communication with the thermocouple and digital controller. The heater may be an electric temperature regulation device such as resistive heater, refrigeration system, or thermoelectric heat pump. Refrigeration systems can cool, and thermoelectric heat pumps can heat or cool as desired. The heater encompasses the chamber 102 (also called the “housing”) that contains the hydrogel. It maintains the hydrogel at a temperature that prevents the nozzle from clogging. It also prevents the hydrogel from premature curing in the chamber 102. The thermocouple is calibrated by measuring the exact temperature of the hydrogel inside the chamber at different input temperatures at the digital controller.

A plurality of meshes 412 may be arranged in series so that the air bubbles that are contained in the hydrogel are reduced in size as the hydrogel passes through each mesh. The pore sizes of the series of meshes 412 may range from 10 to 1000 micrometers. The plurality of meshes are arranged in a similar manner to the plurality of stages depicted in the FIG. 1A. The meshes or micromeshes may be arranged in an alternating fashion with fixture plates as seen in FIG. 3B.

Instead of incorporating the light photocuring unit 408 inside the main body, it was designed as a separate module with a multipart movable arm to facilitate manual adjustment of the direction of the light used for photoinitiation and photocuring. The multipart movable arm is flexible and can be oriented into any desired position (i.e., it can be directed to point in any direction and illuminate the extruded foam). In an embodiment, the light source irradiates the foam with visible light or ultraviolet (UV) light.

Since the UV light intensity is affected by the direction and distance of the light source, this feature helps to make sure the tip of the outlet nozzle is exposed properly to the light, regardless of the shape and size of the nozzles. The light photocuring unit 408 is designed as a plug-in module to be removable in case of using hydrogels that are not photocurable. It is reversibly attachable to the body of the hand-held printer. The photocurable system may or may not be directly connected to the printer.

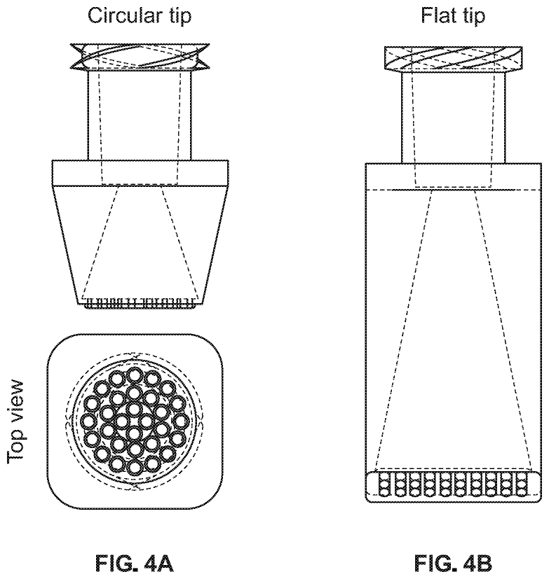

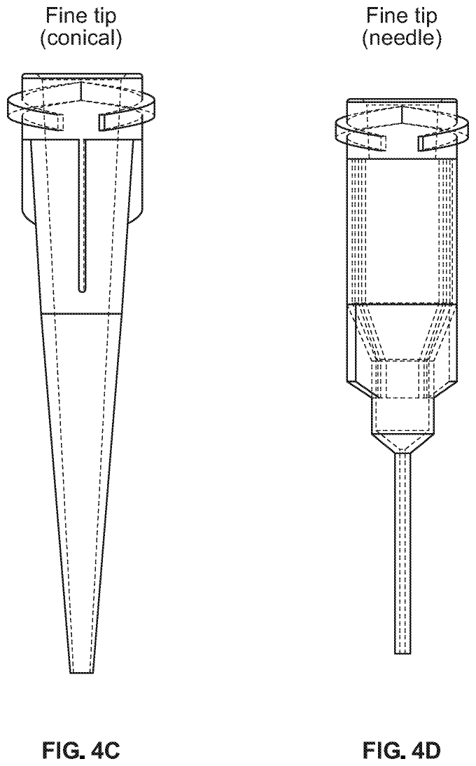

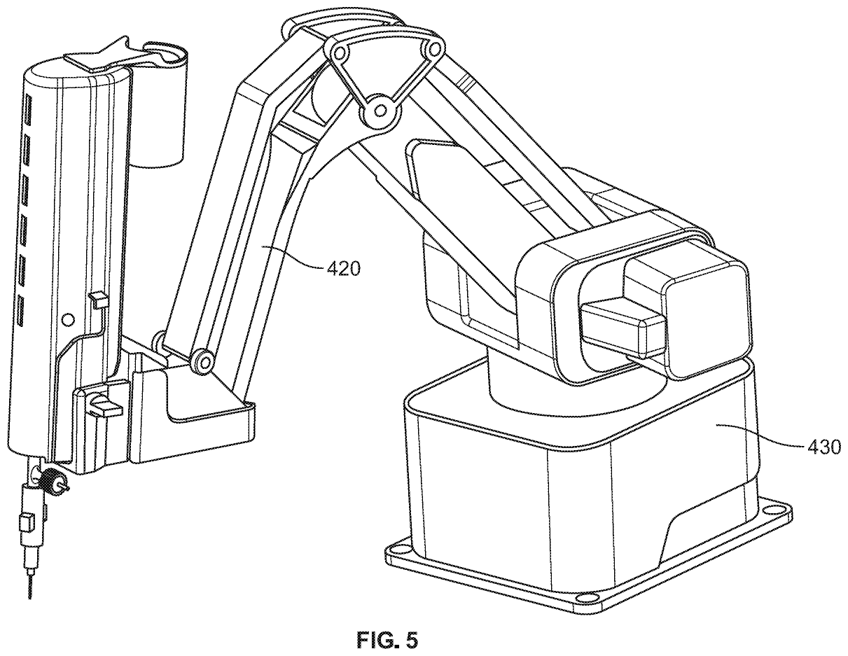

Disclosed herein too are microfluidic nozzles that may be used in conjunction with the printers of the FIGS. 1A, 1B, 2 and 3A-3D. Microfluidic nozzles are fabricated via an assembly of three components. FIGS. 4A-4D include depictions of different exemplary microfluidic nozzles. The first component is a specifically designed three-way connector to introduce air bubbles into the hydrogel stream. This component has two female lure-lock inputs, for hydrogel and air, one male lure-lock output and three small ports for the introduction of air into the hydrogel stream. These ports introduce and mix air into the hydrogel stream more efficiently when compared with a simple three-way connection.

The second component is a tube passage that includes multiple layers of micro-sized meshes, depending on the configuration of use, to split the generated air bubbles into smaller bubble or pores. The configuration of this component, including the size of the meshes and number of mesh layers can be used to manipulate the porosity and pore size distribution of the printed foam.

These parameters, along with the air flow rate, the hydrogel viscosity, temperature, and extrusion rate, can provide a foam having interconnected pores. The microfluidic nozzles when used in conjunction with the printers disclosed herein provide high control over their porosity, average pore size and pore size distribution.

The third component is a male lure-lock end to make the nozzles compatible with different sizes of commercially available printing needles. The outlet tip of the microfluidic nozzles can be either regular needles or custom-designed outlets. The custom designed outlets include a flat flow style (flat tip) to cover a wider area (See FIG. 4B). The other custom designed outlets include a circular shape to cover a relatively wide circular area at once (See FIG. 4A), conical tip (see FIG. 4C) and needle tip (see FIG. 4D).

FIG. 5 is a depiction of a handheld printer configured for use as an automated in-situ printer. The hand-held printer of the FIGS. 3A-3D is integrated with a robot arm using a connector that is operative to lock the printer in place. The printer is reversibly attached to the head of the robot arm. The robot arm 420 is in operative communication with a microprocessor 430 that directs its motion by virtue of directions received from a computer program. This alteration permits the hand-held printer to be used as an automated in-situ printer with minor modifications. This system as shown in the FIG. 5 facilitates consistency of printing when the system is used to reproduce constructs in a mass manufacturing process.

With reference once again to the FIGS. 3A-3D, in one manner of using the printer and the microfluidic nozzles, the air pump 404 blows air flow into the microfluidic nozzles. This induces air bubbles into a stream of GelMA hydrogel in the chamber 402 that is thermally controlled to have a viscosity effective to encapsulate air bubbles. Then the stream of the GelMA hydrogel, containing large air bubbles, passes through the plurality of meshes 412. While the hydrogel phase passes through the meshes, the air bubbles experience shear stresses that force them to split into very small air bubbles or pores within the hydrogel thereby transforming it into a foam. This foam passes through the outlet tip 420 immediately and is in-situ printed onto a site on a living being (that is in need of repair) where it is subjected to crosslinking using the light photocuring unit 408. Since air bubbles within the foam exhibit thermodynamic instability, this step is important as it preserves the fine structure of the in-situ printed foams.

The printer detailed above is exemplified by the following non-limiting examples.

### EXAMPLES

### Example 1

This example is conducted to demonstrate the use of the printer detailed above as well as the porosity and morphology of foams produced by the printer. The hydrogel used is GelMA (which is already described above). Rhodamine (a dye) was used for imaging. The image showed homogeneously distributed pores, with an average pore size ranging from 50 to 100 μm for each print condition.

Morphologically, the pores exhibited a hierarchical structure encompassing both macropores and micropores within the porous structure. Well-interconnected pores were achieved. Additionally, the data showed that changing the print parameters can significantly affect the porosity of the printed foams in a range of 25 to 75 volume percent (vol %). Five parameters that may influence the quality of the printed foam include hydrogel concentration, temperature, number of meshes, pore sizes in the mesh and the ratio of air flow rate to the hydrogel extrusion rate. These are demonstrated in the examples below.

### Example 2

This example was conducted to demonstrate the effect of the parameters listed above-namely hydrogel concentration (i.e., viscosity), temperature, number of meshes, pore sizes in the mesh and the ratio of air flow rate to the hydrogel extrusion rate on foam quality. Viscosity is defined as the internal friction within a fluid due to its molecular cohesion that results in resistance to flow. This resistance to flow can affect the processes of air bubble incorporation into the hydrogel and also affects the passage of air bubbles through the meshes and splitting them. Accordingly, the viscosity and the parameters that drive it, such as concentration and temperature, are useful factors that may be used to determine the quantity and quality of the pores within the printed foams.

For this example, foams were printed with either 10, 15 or 20 weight percent (wt %) GelMA hydrogel concentration at a 32.23° C. hydrogel temperature. Four layers of mesh with a pore size of 56 μm were used to produce pores at an air flow to hydrogel extrusion rate ratio of 1400.

The 10 wt % GelMA concentration has more large pores and wider distribution of pore size when compared with the samples having 15 and 20 wt %.

The data showed that the 10 wt % GelMA hydrogel concentration has a much wider pore size distribution with a larger average pore size than the samples that contained 15 and 20 wt % hydrogel concentrations. The 20 wt % GelMA concentration showed a narrower pore size distribution compared with the 15 wt % concentration sample, along with smaller average and mode pore size.

From the data, it can be seen that pore size distribution and average pore size vary inversely with hydrogel concentration. The pore sizes vary from 20 to 250 micrometers for the compositions having different hydrogel concentrations. The average pore sizes vary from 50 to 120 micrometers.

### Example 3

This example was conducted to determine the effect of temperature on the foam properties. Temperature is a useful parameter that may be used to change the viscosity of the hydrogel and thereby affect the porosity. The 15 wt % GelMA hydrogel was manufactured at different temperatures, 27.23, 32.23 and 37.23° C. using nozzles with 4 meshes of 56 μm pore size at an air flow to hydrogel extrusion rate ratio of 1400.

Porosity measurements showed that increasing the temperature from 27.23° C. to 32.23° C. significantly increased the porosity from 59 vol % to 70 vol %. Porosity therefore increases with the hydrogel temperature at the nozzle.

### Example 4

For this example, foams printed with 15 wt % GelMA hydrogel at 32.23° C. were subjected to printing using either 2, 4, or 8 meshes of 56 μm pore size at an air flow to hydrogel extrusion rate ratio of 1400.

The quantitative measurements of pore size distribution show that despite the difference in the qualitative images, there was not much difference between the 2 and 4 layers of mesh. The use of 8 meshes produced a much larger average pore size and a much wider pore size distribution than when only 2 to 4 meshes were used. Average pore size and pore size distribution therefore increase with an increase in the number of meshes (above 4 meshes) through which the hydrogel is contacted with.

### Example 5

This example is conducted to demonstrate the effect of pore sizes in the meshes. The pore sizes in the meshes are 28, 56 or 112 μm meshes. Foams were printed with 15 wt % GelMA hydrogel at 32.23° C. at an air flow to hydrogel extrusion rate ratio of 1400.

The qualitative images (not displayed here) show almost consistent pore size distribution among all the mesh sizes and a noticeable difference between the porosity of 112 μm with 28 or 56 μm meshes.

Although the 28 μm meshes may apply stronger shear force to the air bubbles compared with the 56 μm meshes, they were not able to induce any noticeable change in the pore size distribution and porosity. A comparison of 4 layers of 56 or 112 μm meshes, revealed that similar to 2 layers of 56 μm meshes, 4 layers of 112 μm meshes may apply lower shear stresses to the air bubbles that lead to significantly lower porosity. On the other hand, contrary to the 2 layers of 56 μm meshes, having 4 layers of 112 μm meshes was able to induce a narrow pore size distribution.

### Example 6

This example was conducted to determine the effect of air flow to the hydrogel extrusion rate ratio. Foams were printed foams using a 1 mL/min hydrogel extrusion rate. The air flow to hydrogel extrusion rate was either 200, 800, or 1400. Quantitative measurements of pore sizes confirmed the presence of different pore size distributions pattern. While the 1400 ratio presented a narrow pore size distribution around an average pore size of about 55 μm, the lower (200 and 800) ratios both presented a much wider pore size distribution and a larger average pore size of about 80 μm.

Further comparison between the 200 and 800 ratios showed that although they have the same average pore size, the 200 ratio has a little larger average pore size due to the presence of large bubbles.

In summary, using the printing device disclosed herein, the porosity varies from 55 to 80 volume percent. The porosity is measured based on the volume and weight measurement of the foam. Volume and weight were physically measured. A foam with higher volume and lower weight has more porosity.

### Example 7

This example was conducted to demonstrate the properties of a) a one-step printed foam as well as b) a foam manufacture by the homogenizer method (a control group). Mechanical and biodegradation characterizations were performed on both foams (a) and (b) as well as on a bulk hydrogel of the same concentration. A cylinder (1 cm height, 1 cm diameter) is manufactured and then compressed at a rate of 0.1 mm/sec. Stress and strain are calculated from a force-displacement graph.

The mechanical compression test data reveals that the foams (a) and (b) have a lower compressive modulus compared with the bulk hydrogel. Both foams (a) and (b) have the same compressive modulus with very low standard deviations. Since mechanical properties of hydrogels are significantly affected by their porosity, this data can also imply that the porosity of the one-step printed foams is the same as foams generated by the homogenizing method.

### Example 8

The one-step, in-situ foam printer is capable of facilitating both hand-held and automated in-situ printing of colloidal highly porous materials. These materials are generated in real-time during the printing process in a well-controlled manner.

The successful generation and simultaneous in-situ printing of a highly porous foam material were achieved by loading the bioink into the cartridge and meticulously controlling its temperature using the integrated temperature control system. This process was facilitated by the integrated extrusion system, air pump, light photo-crosslinker, temperature control module and microfluidic nozzles.

As seen in FIGS. 3A-3D and 5, all these components are enclosed into a compact and ergonomic case that facilitates easy hand holding whilst maneuvering the printer. To simulate the in-situ printing environment, skin tissue models with different artificially structured wounds were acquired. Subsequently, outlet tips were used to seal these minor defects, replicating the in-situ wound-covering process. A distinctive benefit of in-situ printing is its capacity to precisely apply the wound dressing onto irregular and comparatively large areas of the wound. In this regard, a relatively large and irregularly shaped wound model was used to demonstrate the feasibility of accomplishing this capability using the hand-held printer. By maneuvering the in-situ printer, with a flat tip on the wound area, it is possible to deposit foam structures into the wound area and completely cover it with two layers of one-step printed foam.

### Example 9

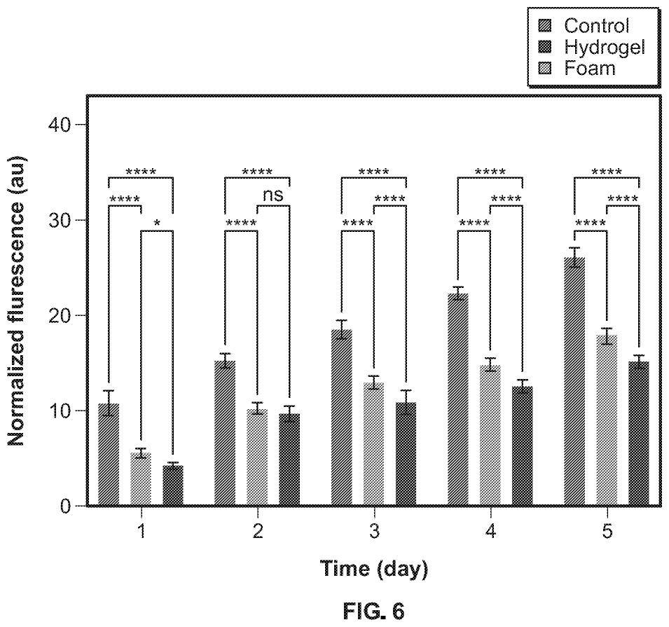

Cell proliferation and viability were assessed over a span of five days using a PrestoBlue metabolic assay, a resazurin cell reduction assay. As is shown in FIG. 6, the fluorescence intensity measurements indicated a consistent linear increase in the metabolic activity of cells subjected to both the one-step printed foam and bulk hydrogel over the course of five days. This trend closely resembled the metabolic rate observed in control cells grown on a 2D cell culture substrate.

In general, the PrestoBlue assay demonstrated a higher proliferation in the one-step printed foam samples compared to the bulk hydrogel, consistently across all time points except for day two where this difference was not significant (FIG. 6). This could result from the porous nature of the one-step printed foam structures, offering increased space for cell growth. This porous framework facilitates efficient nutrient and oxygen transfer, fostering cellular proliferation within 3D constructs. Additionally, the higher surface-area-to-volume ratio of the foam supports cell expansion, eliminating the necessity for scaffold remodeling, and amplifies cellular activity.

Furthermore, the assessment of cell spreading in both the porous one-step printed foam and the bulk hydrogel was conducted. The outcomes underscored a significant enhancement in cell spreading within the one-step printed foams. Conversely, the bulk hydrogel, featuring only nanoscale pores, failed to support cell spreading, and cell areas remained relatively constant. Comparing the microscopic images (not shown) showed the noticeable differences between the structures. The cells cultured on bulk hydrogel are less spread in depth of the structure and covered the surface, on the other hand, one-step printed foams provided the cells with large pores in the structure that help them spread deep into the structure without losing access to nutrition and oxygen.

### Example 10

In order to assess the wound healing efficacy of the printed foams, the wound closure rates in full-thickness wounds were studied using C57BL/6 mice. The mice were randomly assigned to the groups of study and accordingly, their wounds were covered with either bulk GelMA hydrogel or one-step printed GelMA foam or left with no treatment as a control.

The structure of the printed foam is distinguishable from the bulk hydrogel and control group at days 0 and 1. Qualitatively the addition of hydrogel alone hindered the proper wound closure, compared with the no treatment control. On the other hand, the incorporation of pores into the hydrogel through one-step foam printing enabled good wound closure comparable with the control.

To evaluate the efficacy of wound healing quantitatively, the wound closure rate and wound area serve as reliable indicators for predicting complete wound healing.

Accordingly, the wound area was measured for the mice on different days during the in vivo study to obtain these indicators.

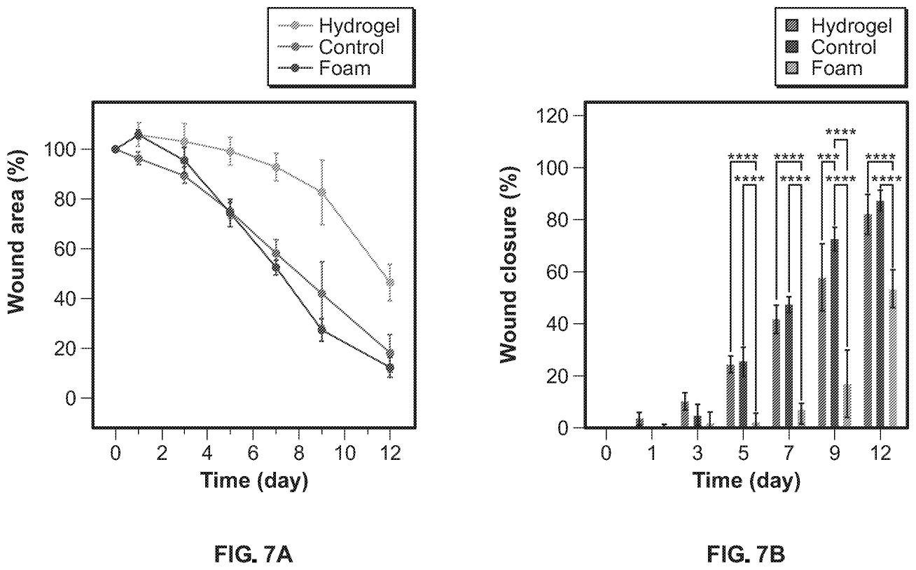

FIG. 7A is a graph that shows that while bulk hydrogel did not provide a high wound closure rate, one-step printed foams showed faster wound closure that resulted in even less wound area, compared with the control, after day 5.

FIG. 7B is a bar graph that shows (from further analytical studies) that the wound closure data reveals that at all time points after day 5, the control and one-step printed foam groups showed significantly better wound closure compared with the bulk hydrogel group. It was also observed that compared with the control group, the one-step printed foam group, showed higher wound closure at all time points after day 5, although this effect was only significant at day 9.

The capacity of the hand-held printer to print the ink directly facilitates in-vivo scaffold printing at the defect site. This approach offers a rapid, straightforward, and controllable treatment method. It also addresses challenges associated with the traditional implantation and fixation of scaffolds by eliminating the need for additional adhesive materials. This innovative technique presents a promising solution for overcoming complexities in the treatment process, showcasing its potential in advancing efficient and effective medical interventions.

While the invention has been described with reference to some embodiments, it will be understood by those skilled in the art that various changes may be made, and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiments disclosed as the best mode contemplated for carrying out this invention, but that the invention will include all embodiments falling within the scope of the appended claims.

1. A printer for manufacturing a construct for use as a prosthetic, the printer comprising:
a housing;
a piston that is operative to reciprocate in the housing to extrude a volume of a hydrogel; and
a stage comprising a porous screen and an inlet port disposed downstream of the housing;
where the inlet port is operative to introduce air into the hydrogel; and where the porous screen is operative to reduce air bubble sizes in the hydrogel.

2. The printer of claim 1, further comprising a driving mechanism in operative communication with the piston and is adapted to impart reciprocating motion to the piston within the housing.

3. The printer of claim 1, where the driving mechanism comprises a stepper motor.

4. The printer of claim 1, where the air is forced into the stage via a motor.

5. The printer of claim 1, where an air flow to hydrogel extrusion rate ratio is 300 to 2000.

6. The printer of claim 1, further comprising a plurality of stages located downstream of the housing; where each stage is operative to introduce one of air, an initiator, a photoinitiator, a crosslinking agent, a filler, an antioxidant, an antiozonant, or a combination thereof into the hydrogel.

7. The printer of claim 6, wherein each stage of the plurality of stages comprises a porous screen and where a porous screen in one stage has a different porosity from another screen in another stage.

8. The printer of claim 7, wherein a porous screen in one stage has a different pore size from another screen in another stage.

9. The printer of claim 1, where the hydrogel further comprises therapeutic cells.

10. The printer of claim 2, further comprising a temperature control system and a light photocuring system; where the temperature control system comprises a heater that surrounds the housing and where the light photocuring system is flexible and can be oriented in any direction.

11. The printer of claim 1, where the light photocuring system emits ultraviolet radiation.

12. The printer of claim 10, where the driving mechanism, the temperature control system and the light photocuring system are in operative communication with a microprocessor.

13. A printer for manufacturing a construct for use as a prosthetic, the printer comprising:
a first syringe;
a second syringe;
a three-way valve in fluid communication with and located downstream of the first syringe and the second syringe; and
a porous screen located in the three-way valve in a fluid pathway between the first syringe and the second syringe; where the mesh is operative to reduce a size of air bubbles contained in a hydrogel or a bioink that is ejected from the first syringe into the second syringe.

14. The printer of claim 13, where the three-way valve is rotated to facilitate a removal of a foam that contains the hydrogel or the bioink from the printer.

15. A method of manufacturing a construct, the method comprising:
blending a hydrogel with air to form an aerated hydrogel;
transmitting the aerated hydrogel through a plurality of porous screens to form a foam;
disposing the foam on a substrate; and
curing the foam with thermal energy or electromagnetic radiation to form the construct.

16. The method of claim 15, further comprising mixing the hydrogel with therapeutic cells.

17. The method of claim 15, wherein the mixing of the hydrogel with the therapeutic cells is conducted after the curing of the foam.

18. The method of claim 15, wherein the blending of the hydrogel with the air is conducted by transmitting the aerated hydrogel between two syringes separated by a porous screen.

19. The method of claim 15, wherein the transmitting of the aerated hydrogel through the plurality of screens is brought about by a driving mechanism.
